(19) United States
(12) Patent Application Publication
(10) Pub. No.: US 2006/0147531 A1
(43) Pub. Date: Jul. 6, 2006

(54) Tamsulosin core with a coating of polyvinylpyrrolidone and polyfinylacetate

(76) Inventors: Mojca Segula, Maribor (SI); Robert Pisek, Ljubljana (SI); Franc Vrecer, Straza (SI); Marjanca Breznik, Ljubljana (SI); Lidia Cernosa, Kostanjevica na Krki (SI); Ivanka Banko, Dvor 18a (SI)

Correspondence Address: COHEN, PONTANI, LIEBERMAN & PAVANE, 551 FIFTH AVENUE, SUITE 1210, NEW YORK, NY, 10176, US

(21) Appl. No.: 10/562,737

(22) PCT Filed: Jun. 30, 2004
(86) PCT No.: PCT/EP04/07131

§ 371 Date: Feb. 21, 2006

(30) Foreign Application Priority Data
Jul. 1, 2003 (DE) 103 29 812.6
Jul. 22, 2003 (DE) 103 33 497.1

(51) Int. Cl. A61K 9/26

(52) U.S. Cl. 424/470

(57) ABSTRACT

The present invention is directed to a sustained/release pharmaceutical composition containing Tamsulosin and having a reduced food effect.

The present invention relates to sustained-release pharmaceutical compositions comprising (−)-(R)-5-{2-[2-(o-ethoxyphenoxy)-ethylamino]-propyl}-2-methoxybenzene-sulfonamide, in the following referred to by its generic name “Tamsulosin”.

Tamsulosin is an α₁-adrenoceptor blocking agent, which exhibits selectivity for α₁-receptors in the human prostate. At least three discrete α₁-adrenoceptor subtypes have been identified: α_{1A}, α_{1B} and α_{1D}, the distribution of which differs between human organs and tissues. Approximately 70% of the α₁-receptors in human prostate are of the α_{1A}-subtype. Therefore Tamsulosin is used for the treatment of the symptoms associated with benign prostatic hyperplasia, such as bladder outlet obstruction, which is comprised of static and dynamic components. The static component is related to an increase in prostate size which is partially caused by proliferation of smooth muscle cells in the prostatic stroma. The severity of benign prostatic hyperplasia symptoms and the degree of urethral obstruction do, however, not correlate well with the size of the prostate. The dynamic component is a function of an increase in smooth muscle tone in the prostate and bladder neck leading to constriction of the bladder outlet. Smooth muscle tone is mediated by the sympathetic nervous stimulation of α₁-adrenoceptors, which are abundant in the prostate, prostatic capsule, prostatic urethra and bladder neck. Blockade of these adrenoceptors can cause smooth muscles in the bladder neck and prostate to relax, resulting in an improvement in urine flow rate and a reduction in symptoms of benign prostatic hyperplasia.

Sustained-release dosage forms are becoming very important for the optimisation of therapy not only because the frequency of administration can be reduced, but also because of the reduction of fluctuations in the blood level. A lower maximum level of the drug in the blood may reduce the severity of dose-dependent side effects and thus may improve the drug products tolerance. Therapeutic efficacy and safety of drugs, which are administered by conventional methods, can be improved by regulating the site and/or the rate of drug delivery. When it is desired to maintain the pharmacological effects of the drug over a long period of time sustained-release pharmaceutical compositions are often the matter of choice. However, sustained-release pharmaceutical compositions containing Tamsulosin have the drawback of exhibiting a strong food effect, i.e. the ratio between maximum plasma concentrations of the Tamsulosin determined after administration of said pharmaceutical composition under fasting conditions and under fed conditions is quite high with values of about 1.7.

Heretofore, only few sustained-release pharmaceutical compositions comprising Tamsulosin have become known.

EP 1 043 031 A1 discloses sustained-release pharmaceutical compositions of ionic pharmaceutically active substances, including Tamsulosin hydrochloride, by adding an equimolar amount of ionic compounds with opposite charge in respect to the ionic pharmaceutically active substance.

EP 0 194 838 B1 discloses granules of from 0.1 to 1.5 mm comprising a mixture of Tamsulosin hydrochloride, crystalline cellulose carrier in an amount of at least 50 solids wt. % and release control agent comprising water and water insoluble macro-molecular substance, selected from acrylic acid series polymers and copolymers and cellulose derivatives that account for up to 30% of the solid components.

Also the Flomax® capsules sold by Boehringer Ingelheim which are described in Physician's Desk Reference, Micromedex(R) Health-care Series Vol. 115 and contain Tamsulosin hydrochloride and methacrylic acid copolymer together with microcrystalline cellulose exhibit a strong food effect, having a ratio between maximum plasma concentrations of the Tamsulosin hydrochloride determined after administration of said pharmaceutical composition under fasting conditions and under fed conditions of about 1.7.

Due to the food effect, which is shown by all the known sustained-release pharmaceutical compositions containing Tamsulosin, persons that are administered said pharmaceutical compositions and are fasted reach much higher levels of Tamsulosin in the blood than persons that are administered said pharmaceutical composition but are fed. This creates a situation in which a person that is fasted may suffer from adverse effects, whereas a person that is fed might not realize the desired effect at all, even though they both were administered the same amount of Tamsulosin.

It is clear from the above that this food effect in the existing pharmaceutical compositions is a strong disadvantage. Therefore, there exists a need for an improved sustained-release pharmaceutical composition that overcomes this drawback.

This object is surprisingly solved by the sustained-release pharmaceutical composition according to claims 1 to 21 and 22 to 25. The invention is also directed to a process for the manufacture of said composition according to claims 26 and 27.

The sustained-release pharmaceutical composition according to the invention is characterized in that it comprises a core of

a) Tamsulosin in the form of the base and/or in the form of a pharmaceutically acceptable salt, and

b) optionally at least one excipient and

c) a coating which contains a combination of polyvinylacetate (PVAC) and polyvinylpyrrolidone (PVP).

The Tamsulosin (a) can be used in the form of the base, i.e. in the form of (—)-(R)-5-{2-[2-(o-ethoxyphenoxy)-ethylamino]-propy-1}-2-methoxybenzene-sulfonamide, and/or in the form of a pharmaceutically acceptable salt, e.g. hydrochloride, hydrobromide, phosphate, nitrate, sulfate, organic sulfonate, acetate, propionate, oxalate, malonate, succinate, glutarate, tartrate, maleate, besilate, and the like. It is a preferred embodiment of the present invention that the pharmaceutically acceptable salt is Tamsulosin hydrochloride.

In a preferred embodiment the amount of Tamsulosin in the pharmaceutical composition according to the invention ranges from 0.005 to 1.20 wt. %, preferably 0.05 to 0.6 wt. %, more preferably 0.08 to 0.20 wt. %, based on the total weight of the pharmaceutical composition.

It is another preferred embodiment that a single dosage form of the pharmaceutical composition according to the invention contains Tamsulosin in an amount of from 0.025 to 1.2 mg.

The preferred amounts of Tamsulosin administered as daily dosage are 0.05 to 1.2 mg.

The pharmaceutical composition according to the invention is preferably in the form of tablets, capsules, granules or pellets.

These tablets, capsules, granules or pellets can be administered to a patient in need thereof one or two times daily. It is preferred to administer said pharmaceutical composition as seldom as possible, i.e. a once daily administration is most preferred. In addition to the Tamsulosin the cores can preferably comprise one or more excipients (b) such as neutral pellets, an embedding material for the Tamsulosin, non-ionic surfactant(s) and other adjuvants such as e.g. antisticking agents.

Neutral pellets can be made of any material which is pharmaceutically acceptable but are preferably made from starch and/or sucrose. They can have each size which is in accordance with the specified use, i.e. the pharmaceutical composition which is based on said neutral pellet has to be swallowable. The size of said pellets therefore preferably ranges from 0.01 to 10 mm, with a range of from 0.1 to 1 mm being preferred, a range of from 0.6 to 0.9 mm being particularly preferred and a size of from 0.7 to 0.85 mm being most preferred. The neutral pellets can account for 30 to 90 wt. % of the pharmaceutical composition according to the invention, in particular the amount of said pellets may range from 35 to 80 wt. % and preferably from about 45 to about 70 wt. % of said pharmaceutical composition.

The embedding material for the Tamsulosin can be any material that is appropriate for storage and release of Tamsulosin, i.e. which is substantially inert in respect to Tamsulosin. In a preferred embodiment the embedding material for the Tamsulosin of the pharmaceutical composition according to the invention is selected from the group consisting of polyvinyl pyrrolidone and cellulose ethers, such as hydroxypropyl cellulose or hydroxypropylmethyl cellulose. Pharmaceutical compositions according to the invention, wherein the embedding material is cellulose ether are even more preferred. The embedding material is mixed with the Tamsulosin and then either formed to pellets or tablets, followed by coating with coating (c), or the mixture of embedding material and Tamsulosin is coated onto neutral pellets as defined above, followed by application of coating (c).

It is also preferred that the weight ratio of Tamsulosin to the embedding material in the pharmaceutical composition according to the invention ranges from 1:3 to 1:25, in particular from 1:4 to 1:19, with a range of from 1:10 to 1:18 being most preferred.

The cores of the pharmaceutical composition according to the invention can further comprise one or more non-ionic surfactants selected from the group consisting of alkylglycosides, alkylmaltosides, alkylthioglucosides, polyoxyethylene alkyphenols, polyoxyethylene alkylethers, polyethylene glycol fatty acid esters, polyethyleneglycol glycol glycerol fatty acid esters, polyoxyethylene-polyoxypropylene block copolymers, polyglyceryl fatty acid esters, polyoxyethylene glycerides, polyoxyethylene vegetable oils, polyoxyethylene hydrogenated vegetable oils and sterols.

Such surfactants are preferably included in a coat which also contains Tamsulosin, they are the other way round usually not found e.g. in a subcoat.

A pharmaceutical composition according to the invention, wherein the non-ionic surfactant is a polyoxyethylene sorbitan fatty acid ester is even more preferred.

A pharmaceutical composition according to the invention, wherein the weight ratio of Tamsulosin to the non-ionic surfactant(s) ranges from 1:12 to 1:25, preferably from 1:14 to 1:22 and in particular from 1:16 to 1:19, is preferred.

An antisticking agent, such as talc, can also be used in the core. It reduces the sticking tendency and thereby prevents agglomeration of cores as well as adhesion effects to the wall during a coating process.

Such an antisticking agent can be present in the core of the pharmaceutical composition according to the invention in an amount of 0.1 to 10 wt. %, preferably 0.5 to 5 wt % and most preferably 1 to 4 wt. % of the pharmaceutical composition.

In case neutral pellets are present in the core of the pharmaceutical composition according to the invention a sub-coat can also be present in the core in addition to the components described above. The function of said sub-coat is to separate the neutral pellet from the Tamsulosin or from the layer that contains Tamsulosin. This sub-coat, as all the possible sub-coats in this invention, preferably comprises water soluble substances or polymers, e.g. polyvinyl alcohol or cellulose derivatives. According to the invention each possible sub-coat represents preferably 1 to 5 wt %, in particular 1 to 3 wt % of the pharmaceutical composition.

In a preferred embodiment of the invention the amount of polyvinyl acetate and polyvinyl pyrrolidone in the coating (c) in the pharmaceutical composition ranges from 1 to 30 wt. %, in particular 2 to 20 wt. % and preferably 3 to 10 wt. % based on the total weight of the pharmaceutical composition.

It is also preferred that the coating (c) has a layer thickness in the range of 10 to 50 μm, particularly preferred 10 to 40 μm and even more preferred 10 to 25 μm.

The release rate from these coated pharmaceutical compositions decreases with increasing thickness of the coating layer. Initially, water has to penetrate the coating and enter the particle in order to at least partially dissolve the Tamsulosin before the Tamsulosin can diffuse out through the coating.

The coverage by the coating layer should not be less than 1.0 mg per cm² (which corresponds to a thickness of about 10 μm) since otherwise film defects and burst effects are to be expected.

The coating (c) which contains polyvinyl acetate (PVAC) and polyvinyl pyrrolidone (PVP) as a polymer combination with a sustained-release effect may further contain e.g. a plasticizer and an antisticking agent.

The coating can be produced by preferably using a preformulated mixture, e.g. a dispersion of polyvinyl acetate and polyvinyl pyrrolidone, which may contain surface active substances, such as polysorbates, sodium dioctyl sulfosuccinate, sodium lauryl sulfate, polyoxyethylenepropylene copolymers for stabilization of the coating dispersion.

In a preferred embodiment, the ratio of polyvinyl acetate to polyvinyl pyrrolidone in the coating layer (c) of the pharmaceutical composition according to the invention ranges from 1.5:1 to 14:1 based on weights, in particular from 5:1 to 13:1. A pharmaceutical composition wherein the ratio of polyvinyl acetate to polyvinyl pyrrolidone in said layer ranges from 9:1 to 12:1 based on weights is even more preferred.

Such preferred combinations of polyvinyl acetate and polyvinyl pyrrolidone possess an enormous plasticity. As a result, film coatings based on these combinations are very resistant to mechanical stress and show a self-repair mechanism, particularly when a coated particle is introduced into an aqueous medium. Because of this self-repair mechanism and the overall stability, the probability of an instantaneous release of the Tamsulosin is reduced and therefore the safety of the pharmaceutical composition is further enhanced. It is a further benefit that a curing step which is necessary for other commercially available polymer combinations is optional and not obligatory.

The optional plasticizer enhances film formation and the flexibility of the coating. Suitable plasticizers are 1,2-propylene-gylcol, triethylcitrate, polyethylene glycols and triacetin. The preferred plasticizer is triethylcitrate.

It is preferred that the amount of plasticizer in the polyvinyl acetate and polyvinyl pyrrolidone-containing coat (c) is in the range of 0 to 15 wt. %, preferably 5 to 13.5 wt. % and most preferred 8 to 12 wt. % based on the weight of the combination of polyvinyl acetate and polyvinyl pyrrolidone.

An antisticking agent, such as talc, can also be used in the coating. It reduces the sticking tendency and thereby prevents agglomeration of pellets as well as adhesion effects to the wall during the coating process.

It is preferred that the weight ratio of the antisticking agent in said coat to the dry weight of a preformulated mixture of polyvinyl acetate and polyvinyl pyrrolidone is at least 1:1 and up to 1:50 and preferably at least 1:2 and up to 1:20 and most preferably is at least 1:2.5 and up to 1:10, with a value of about 1:3 being most preferred.

The release from the compositions according to the invention can not only be controlled by the thickness of the coating which contains polyvinyl acetate and polyvinyl pyrrolidone, but also by an optional overcoat, such as an enteric coating. An enteric coating contains an enteric polymer and other pharmaceutically acceptable compounds. Suitable enteric polymers are acrylic acid polymers and copolymers, cellulose derivatives and polyvinyl acetatephthalate. Suitable pharmaceutically acceptable compounds are plasticizers, e.g. triethyl citrate, polyethylene glycols or triacetin, antisticking agents, e.g. talc, pigments, e.g. titanium dioxide, and sodium carboxymethylcellulose.

In a preferred embodiment the amount of the enteric coat in a pharmaceutical composition according to the invention is in the range of 10 to 60 wt. %, preferably 12.5 to 45 wt. % and most preferably about 15 to about 30 wt. % of said pharmaceutical composition.

In cases where an enteric coating is present a sub-coating is preferably used to separate the PVP/PVAC polymer combination from the enteric polymer to avoid any interactions between these layers.

It is preferred that the pharmaceutical composition according to the invention further comprises one or more antioxidants selected from the group consisting of alkyl gallates (e.g. dodecyl-, ethyl-, octyl-, propyl-gallate), butylated hydroxyanisole, butylated hydroxytoluene, tocopherols (e.g. alpha tocopherol), ascorbic acid palmitate, ascorbic acid, sodium ascorbate, potassium and sodium salts of sulphurous acid (e.g. bisulphites, metabisulphites, sulphites), flavonoides (rutin, quercetin).

Such antioxidants can be present in the core, preferably in the coat which contains the Tamsulosin. It is preferably present in an amount of 0.005 to 1 wt %, preferably 0.01 to 0.1 wt. % and most preferably from about 0.025 to about 0.08 wt % of the pharmaceutical composition.

A pharmaceutical composition according to the invention, wherein the antioxidant is ascorbic acid or butylated hydroxyanisole is preferred.

A pharmaceutical composition according to the invention, wherein the weight ratio of Tamsulosin to the antioxidant(s) ranges from 20:1 to 1:5, in particular from 10:1 to 3:1, is preferred.

It is preferred that the pharmaceutical composition according to the invention further comprises one or more organic acid/acids, such as citric acid, which is/are used to maintain the pH of the coating suspension which contains the Tamsulosin at values of about 3 and therefore is preferably present in said coat. It is preferably present in an amount of 0.005 to 1 wt %, preferably 0.01 to 0.1 wt. % and most preferably 0.025 to 0.05 wt % of the pharmaceutical composition.

It is preferred that the pharmaceutical composition according to the invention further comprises a chelating agent selected from the group consisting of ethylenediamine tetraacetic acid (edetic acid or EDTA) and edetate salts, e.g. dipotassium edetate, disodium edetate, edetate calcium disodium, sodium edetate, trisodium edetate. A pharmaceutical composition according to the invention, wherein the weight ratio of Tamsulosin to the chelating agent ranges from 50:1 to 1:1, in particular 20:1 to 1.5:1 is preferred. A pharmaceutical composition according to the invention wherein the chelating agent is sodium edetate is even more preferred.

According to one embodiment the pharmaceutical compositions of the invention simply consist of a core made from Tamsulosin and preferably an embedding material, and a coat made of PVAC/PVP.

Embodiments in which a neutral pellet is present and the Tamsulosin together with the embedding material as well as the PVAC/PVP coat are assembled layerwise around said neutral pellet are preferred. It is particularly preferred that additional layers which act as sub-coats or as enteric coat are present.

Accordingly a particularly preferred embodiment of a pharmaceutical composition according to the invention has the following configuration:

neutral pellet

preferably first sub-coat

Tamsulosin-containing coat

preferably second sub-coat

PVAC/PVP-coat

preferably third sub-coat

enteric coat.

These various coats are applied layerwise onto the neutral pellet. The PVAC/PVP- or the enteric coat is located on the outside of the pharmaceutical composition.

The invention also relates to a sustained-release pharmaceutical composition which comprises Tamsulosin in the form of the base and/or in the form of a pharmaceutically acceptable salt which is characterized in that the ratio between maximum plasma concentrations of Tamsulosin determined after administration of said pharmaceutical composition under fasting conditions and under fed conditions according to the Cmax_{fasting/fed}-test is less than 1.35. This is advantageous since the difference in the Tamsulosin blood level of patients that are fed and patients that are fasted is minimized.

The Cmax_{fasting/fed}-test is described in Example 8.

In the preferred compositions the ratio is less than 1.25, preferably less than 1.2 and most preferred is a pharmaceutical composition wherein the ratio is equal to or less than 1.15.

A sustained-release pharmaceutical composition, comprising a core of

(a) Tamsulosin in the form of the base and/or in the form of a pharmaceutically acceptable salt, and

(b) optionally at least one excipient and

(c) a coating which contains a combination of polyvinylacetate and polyvinyl pyrrolidone

which pharmaceutical composition exhibits a ratio between maximum plasma concentrations of the Tamsulosin determined after administration of said pharmaceutical composition under fasting conditions and under fed conditions according to the Cmax_{fasting/fed}-test of less than 1.35 represent one of the preferred embodiments of the invention.

The present invention also relates to a process for the manufacture of a pharmaceutical composition according to the invention comprising the steps of

(i) providing Tamsulosin (a), optionally in combination with at least one excipient (b) and

(ii) applying a coating (c) thereon which contains a combination of polyvinyl acetate and polyvinyl pyrrolidone.

In a preferred embodiment the process comprises the steps of

(i1) providing neutral pellets which preferably are made of sucrose and maize starch or microcrystalline cellulose,

(i2) optionally coating said neutral pellets with a first subcoat,

(i3) coating said pellets of step (i1) or (i2) with a dispersion that contains Tamsulosin, either in water or organic solvent, preferably alcohol, or a mixture of water and alcohol,

(i4) optionally coating said pellets of step (i3) with a second subcoat,

(ii1) applying on said pellets of steps (i3) or (i4) a coating which contains a combination of polyvinyl acetate and polyvinyl pyrrolidone, and

(ii2) optionally coating said pellets of step (ii1) with a third subcoat, and

(ii3) optionally coating said pellets of step (ii1) or (ii2) with an overcoat.

The coating of the pellets in step(s) (i2), (i3), (i4), (ii1), (ii2) and/or (ii3) is preferably performed utilizing conventional methods known in the art. For example the coating can be applied in a fluidized bed or coating pan.

The pharmaceutical composition according to the invention is preferably produced using a fluidized bed coater, e.g. a bottom, a tangential or a top spray coater. A fluidized bed system, e.g. Wurster, Hüttlin, is one in which an airjet, injected from underneath, fluidizes the particles and effects a drying while the coating material is sprayed. The individual coated particles, e.g. granules or pellets, thus obtained can be used for formation of units such as capsules or tablets.

Such a production process is very efficient and economical reasonable because all phases of the manufacturing, such as coating and drying, take place in a single pot, e.g. a fluidized bed coater. Further, such a production process leads to compositions wherein the reproducibility of the release characteristics of Tamsulosin is excellent.

Said processes lead to:

sustained-release pharmaceutical compositions, comprising a core of

(a) Tamsulosin in the form of the base and/or in the form of a pharmaceutically acceptable salt, and

(b) optionally at least one excipient and

(c) a coating which contains a combination of polyvinyl acetate and polyvinyl pyrrolidone,

as well as to those which comprise Tamsulosin in the form of the base and/or in the form of a pharmaceutically acceptable salt and exhibit a ratio between maximum plasma concentrations of the Tamsulosin determined after administration of said pharmaceutical composition under fasting conditions and under fed conditions according to the Cmax_{fasting/fed}-test of less than 1.35.

Prefered is a process which leads to sustained-release pharmaceutical compositions having the following composition:

preferably a neutral pellet, which preferably is made of sucrose and maize starch or microcrystalline cellulose in an amount of 30 to 90 wt %,

preferably a first subcoat in an amount of 1 to 5 wt %,

a coat in an amount of 1 to 20 wt % that contains Tamsulosin,

preferably a second subcoat in an amount of 1 to 5 wt %,

a coat in an amount of 5 to 35 wt % which contains a combination of polyvinyl acetate and polyvinyl pyrrolidone,

preferably a third subcoat in an amount of 1 to 5 wt % and

preferably an overcoat such as an enteric coat in an amount of 10 to 60 wt %,

with the sum of all components adding up to 100 wt %.

The pharmaceutical composition according to the invention is preferably in the form of tablets, capsules, granules or pellets. Tablets, granules or pellets can be obtained directly by the processes described above. Capsules can be obtained by encapsulating said granules or pellets. Alternatively tablets can be produced by pressing the granules or pellets obtained by the processes described above into tablets on standard machinery. Tabletting aids can be used to facilitate the process.

The Tamsulosin-release profile of the pharmaceutical composition can be tailored by varying the size of the granules or pellets, the thickness of coat (c) or by combining granules or pellets having different release profiles. The different pellets or granules can be combined within a capsule or can be formed to tablets together with a disintegrant which causes the tablet to decompose into single pellets or granules after swallowing. This possibility of tailoring the release profile by using different granules or pellets is an advantage over tablets that use sustained release matrix materials as disclosed e.g. in WO 03/039531.

The invention is further illustrated with reference to the following Examples.

### EXAMPLES

### Example 1

Neutral pellets, made from starch and sucrose (d=0.71 to 0.85 mm, 3200.0 g), representing cores, were coated with a dispersion which contained 7.04 g of Tamsulosin hydrochloride, 119.86 g polyvinyl pyrrolidone, 126.72 g polyethylene 20 sorbitan monooleate as non-ionic surfactant, 123.20 g of talc and 2002.00 g of water. The dispersion was prepared as follows:

The total quantity of water was added to a suitable container and stirred using a propeller stirrer or a similar type of stirrer. First, polyethylene 20 sorbitan monooleate was dissolved in the water at 40° C., then Tamsulosin hydrochloride was added and dissolved. Afterwards, polyvinyl pyrrolidone was added and dissolved, and subsequently talc was added. To avoid settling, stirring was continued throughout the coating process.

The cores were coated by spraying using a Wurster fluidized-bed coater.

The coated cores were subsequently coated with a dispersion that contained 24.19 g triethylcitrate, 86.39 g talc, 806.34 g water and 806.34 g of a water dispersion which contained 30 wt. % of a polymer combination of PVP and PVAC in a ratio of 1:10.8. This coating dispersion was prepared as follows:

The total quantity of water was added to a suitable container and stirred using a propeller stirrer or a similar type of stirrer. First, triethylcitrate was added to the water, followed by addition of talc and of the dispersion of PVP and PVAC. Mixing was continued for 10 minutes, after which time the dispersion was completed. To avoid settling, stirring was continued throughout the coating process.

The cores containing the Tamsulosin were coated by spraying using a Wurster fluidized-bed coater.

### Example 2

Cores (neutral pellets: d=0.71 to 0.85 mm; 2660.0 g) were coated with a dispersion that contained 6.44 g Tamsulosin hydrochloride, 109.48 g polyvinyl pyrrolidone, 115.92 g non-ionic surfactant polyethylene 20 sorbitan monooleate, 112.70 g talc and 1831.95 g water. The dispersion was prepared as follows:

The total quantity of water was added to a suitable container and stirred using a propeller stirrer or a similar type of stirrer. First, polyethylene 20 sorbitan monooleate was dissolved in the water at 40° C. Then Tamsulosin hydrochloride was added and dissolved. Afterwards polyvinyl pyrrolidone was added and dissolved followed by the addition of the talc. To avoid settling, stirring was continued throughout the coating process.

The cores were coated by spraying using a Wurster fluidized-bed coater.

The coated cores were then coated with a dispersion that contained 19.22 g triethylcitrate, 68.46 g talc, 640.51 g water and 640.51 g of the water dispersion which contained 30 wt. % of a polymer combination of PVP and PVAC in a ratio of 1:10.8. This coating dispersion was prepared as follows:

The total quantity of water was added to a suitable container and stirred using a propeller stirrer or a similar type of stirrer. First triethylcitrate, and then talc and the PVAC/PVP dispersion were added to the water. Mixing was continued for 10 minutes, after which time the dispersion was completed. To avoid settling, stirring was continued throughout the spraying process.

The Tamsulosin containing cores were coated by spraying using a Wurster fluidized-bed coater.

The resulting particles having a Tamsulosin containing layer and a PVAC/PVP-containing layer were then coated with a sub-coat. The dispersion for spraying a sub-coat contained 94.98 g Opadry II HP White® which is a polyvinyl alcohol product and 379.90 g water. The sub-coating dispersion was prepared as follows:

The total quantity of water was added to a suitable container and stirred using a propeller stirrer or a similar type of stirrer, such that a liquid vortex was just produced without to much air being drawn into the liquid. The Opadry was added at the fastest possible rate, i.e. without incurring powder flotation, to the vortex. Due to a rise in suspension viscosity as the addition of the Opadry proceeds it may be necessary to increase the stirrer speed in order to maintain a vortex. After all the Opadry was added, the stirrer speed was reduced until the vortex was just eliminated and stirring was continued for at least 45 minutes, after which time the dispersion was completed. To avoid settling, stirring was continued throughout the spraying process.

The particles were coated using the Wurster fluidized-bed coater.

These coated particles having a layer containing Tamsulosin, a layer containing PVAC/PVP-combination and a layer containing the Opadry® were subsequently coated with an enteric coating. The coating dispersion contained 46.17 g triethylcitrate, 212.87 g talc, 57.91 g titanium dioxide, 18.31 g sodiumcarboxy methylcellulose, 18.31 g polyethylene glycol 6000, 1721.71 g water and 1565.20 g Eudragit L 30 D-55® dispersion which contained 30.0 wt %, i.e. 469.56 g, of dry acrylic polymer.

The preparation of the enteric coating dispersion was performed as follows:

Manufacturing of the pigment solution dispersion was performed by dissolving polyethylene glycol in the required amount of water, adding talc afterwards and stirring constantly with the homogenizing equipment with titanium dioxide, triethylcitrate and sodiumcarboxy methylcellulose being incorporated and homogenized for 15 minutes.

Preparation of the Eudragit dispersion was performed by diluting the given quantity of Eudragit L 30 D-55® with water to result in a solids content of about 20% by weight.

Manufacturing of the enteric coating dispersion was performed by adding said pigment slurry to said diluted Eudragit dispersion while stirring.

To avoid settling, stirring was continued throughout the spraying process.

The particles were coated using a Wurster fluidized-bed coater.

### Example 3

Neutral pellets, made from starch and sucrose (d=0.71 to 0.85 mm, 2660.0 g), were coated with a first subcoat as a protective layer. The dispersion for this first subcoat contained 70.80 g Opadry II HP White® and 319.20 g water. The preparation of this subcoating dispersion was as follows:

The total quantity of water was added to a suitable container and stirred using a propeller stirrer or a similar type of stirrer, such that a liquid vortex was just produced without too much air being drawn into the liquid. The Opadry was added at the fastest possible rate, i.e. without incurring powder flotation, to the vortex. It may be necessary to increase the stirrer speed in order to maintain a vortex as addition of the Opadry proceeds resulting in a rise in suspension viscosity. After all the Opadry had been added, the stirrer speed was reduced until the vortex was just eliminated and stirring was continued for at least 45 minutes, after which time the dispersion was completed. To avoid settling, stirring was continued through out the spraying process.

The particles were coated using a Wurster fluidized-bed coater.

Such precoated cores were then coated by the procedure of example 2.

### Example 4

Neutral pellets, made from microcrystalline cellulose (d=0.71 to 1.0 mm, 2660.0 g), were coated by the procedure of example 1.

### Example 5

Neutral pellets, made from microcrystalline cellulose (d=0.71 to 1.0 mm, 2660.0 g), were coated by the procedure of example 2.

### Example 6

Neutral pellets, made from starch and sucrose (d=0.71 to 0.85 mm; 1900.0 g), represented the cores which were coated with a dispersion which contained 4.40 g of Tamsulosin hydrochloride, 28.60 g of Klucel EF® (hydroxypropyl cellulose), 1.21 g of citric acid, 77.0 g of talc and 1030.70 g of water.

Preparation of the dispersion: the half quantity of water was added to a suitable container and stirred using a propeller or similar type of stirrer. First, citric acid was dissolved in the water, then Tamsulosin hydrochloride was added and dissolved. The second half quantity of water was added to a suitable container and stirred using a propeller or similar type of stirrer. First, Klucel EF® was dissolved in the water, then talc was added. Subsequently, the solution of citric acid and Tamsulosin hydrochloride was added to the suspension of Klucel EF and talc.

To avoid settling, stirring must be continued throughout the spraying process.

The cores were coated using Wurster fluidized-bed coater.

These coated particles having a layer containing Tamsulosin were then coated with a PVAC/PVP layer, Opadry® layer and subsequently with an enteric coating by the procedure of example 2.

### Example 7

Neutral pellets, made from starch and sucrose (d=0.71 to 0.85 mm; 1900.0 g), represented the cores which were coated with a dispersion which contained 4.40 g of Tamsulosin hydrochloride, 28.60 g of Klucel EF® (hydroxypropylcellulose), 2.20 g of ascorbic acid, 77.0 g of talc and 1030.70 g of water.

Preparation of the dispersion: the half quantity of water was added to a suitable container and stirred using a propeller or similar type of stirrer. First, ascorbic acid was dissolved in the water, then Tamsulosin hydrochloride was added and dissolved. The second half quantity of water was added to a suitable container and stirred using a propeller or similar type of stirrer. First, Klucel EF® was dissolved in the water, then talc was added. Subsequently, the solution of ascorbic acid and Tamsulosin hydrochloride was added to the suspension of Klucel EF® and talc.

To avoid settling, stirring must be continued throughout the spraying process.

The cores were coated using Wurster fluidized-bed coater.

These coated particles having a layer containing Tamsulosin were then coated with a PVAC/PVP layer, Opadry® layer and subsequently with an enteric coating by the procedure of example 2.

### Example 8

Absorption of Tamsulosin hydrochloride from 0,4 mg Tamsulosin pellets (Example 2) which were in the form of capsules was tested in a single dose study under fasting and under fed conditions. Differences in Tamsulosin pharmacokinetics between the two conditions were not significant.

For determining the Cmax_{fasting/fed}-ratio the following test was used:

Cmax_{fasting/fed}-Test

A single dose of the tested pharmaceutical composition containing Tamsulosin (0,4 mg) was administered to healthy volunteers aged between 20-30 years, with body weight not more than 10% above or below the ideal weight, non-smokers and with no history of alcohol and drug abuse. Taking prescription medication within the previous 4 weeks or over-the-counter medication within the previous 2 weeks, clinically significant history of reaction to drugs in the past and participation in other clinical test or blood donation within the previous 3 months were considered as criteria not allowing the subject to participate in the test.

A single dose of the tested composition comprising 0.4 mg Tamsulosin were administered to volunteers under fasting and under fed conditions. All volunteers were fasting for 10 hours during the night prior the drug administration. For all subjects water was allowed ad libitum until 2 hours pre-dose. The fed group was supplied with a high-fat breakfast, which consists of 1 fried egg, bacon (1 slice), liver paté (30 g), butter (20 g), white bred (2 slices), 1 roll, milk (3,2% fat, 200 ml) and apple juice (200 ml). This breakfast was supplied half an hour before the drug administration. The fasting group was not supplied with a high-fat breakfast and remained fasting till 4 hours post-dosage. In both groups fluid intake was controlled and consistent for all subjects for the first 4 hours following the drug administration as follows: The drug was given with 200 ml of tap water, at 2 hours post-dose 200 ml of non-carbonated, non-xanthine and non-grapefruit containing soft drink were provided, and water was allowed ad libitum after 4 hours post-dose. At 4 and 10 hours post-dose standardized identical meals (with a non-carbonated, non-xanthine and non-grapefruit containing beverage) were provided to all test subjects.

Blood samples were taken at 0 (pre-drug administration) 1.0, 2.0, 3.0, 4.0, 5.0, 6.0, 7.0, 8.0, 10.0, 12.0, 24.0, 36.0, 48.0 and 60.0 hours post-drug. Then the Cmax values for both groups were determined directly from the individual plasma concentration/time data. Plasma concentrations of Tamsulosin were measured with a LC-MS/MS method.

Said method involved liquid-liquid extraction with diethyl ether followed by reversed-phase HPLC separation with tandem mass spectrometric detection: To plasma samples 100 μl working solution of internal standard were added. Samples were extracted with diethyl ether. The organic phase extracts were evaporated to dryness and redissolved in mobile phase for LC-MS/MS analysis.

Using the obtained data the ratio Cmax_{fasting/fed} was computed to finalize the test.

Other pharmacokinetic parameters were determined by using the same test as above on the basis of individual plasma concentration/time profiles using a model-independent approach, i.e. the parameters were determined directly from measured concentrations without using a mathematical model, which could be developed for a concentration/time profile.

The other parameters were values for the area under the plasma concentration-time curve from time 0 to infinity (AUC), the area under the plasma concentration-time curve from time 0 to the time of last quantifiable concentration (AUCO_t), the peak plasma concentration (Cmax), the mean residence time (MRT), the time to reach Cmax (tmax) and the terminal plasma elimination half life (t1/2). Parameters determined under fasting conditions were compared with those determined under fed conditions. Differences between the two administrations in dependence of the food intake were determined by means of a two-independent-sample t test at 5% significance level.

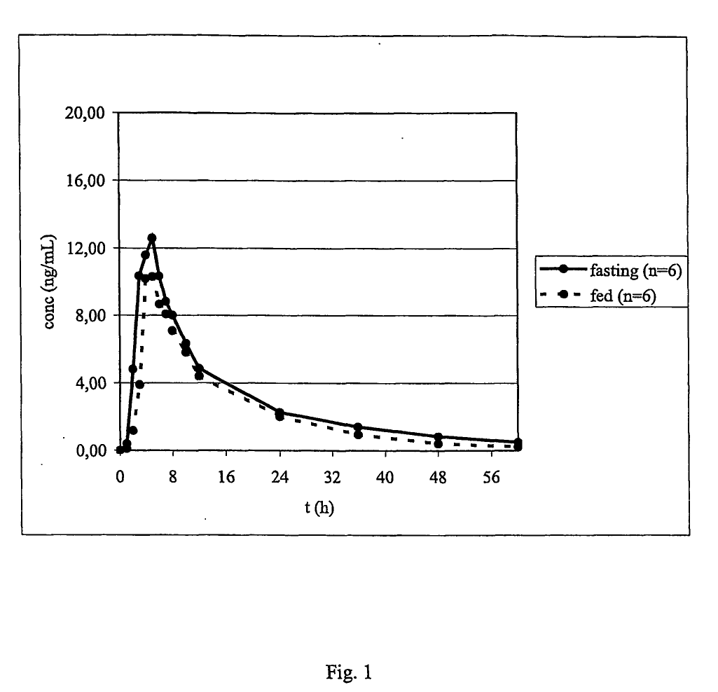

Results are summarized in the following Table 1 and FIG. 1.

TABLE 1

| | AUC | AUCO_t | Cmax | | | |
|---|---|---|---|---|---|---|
| | (ng/ml * h) | (ng/ml * h) | (ng/ml) | MRT (h) | tmax (h) | t½ (h) |
| fasting | 185.23 ± 114.56 | 173.75 ± 100.01 | 13.42 ± 3.50 | 16.84 ± 7.04 | 4.33 ± 0.82 | 11.33 ± 4.33 |
| (n = 6) | | | | | | |
| fed | 141.96 ± 44.00 | 136.22 ± 44.72 | 11.67 ± 3.16 | 16.97 ± 2.29 | 5.17 ± 0.98 | 11.13 ± 1.65 |
| (n = 6) | | | | | | |
| p | 0.41* | 0.42* | 0.39* | 0.97* | 0.14* | 0.92* |

*non significant difference, significance level α = 0.05
p is the probability associated with the t test

The above data show that the composition according to the invention has the advantage of a minimized food effect.

We claim:

1. A sustained-release pharmaceutical composition comprising a core comprising
(a) Tamsulosin in the form of the base and/or in the form of a pharmaceutically acceptable salt, and
(b) optionally at least one excipient and
(c) a coating which contains a combination of polyvinylacetate (PVAC) and polyvinylpyrrolidone (PVP).

2. The pharmaceutical composition according to claim 1, wherein the pharmaceutically acceptable salt is Tamsulosin hydrochloride.

3. The pharmaceutical composition according to claim 1, which comprises Tamsulosin in an amount of 0.005 to 1.20 wt. %, based on the total weight of the pharmaceutical composition.

4. The pharmaceutical composition according to claim 3, which comprises Tamsulosin in an amount of 0.08 to 0.20 wt. %, based on the total weight of the pharmaceutical composition.

5. The pharmaceutical composition according to claim 4, which is a single dosage form and comprises Tamsulosin in an amount of from 0.025 to 1.2 mg.

6. The pharmaceutical composition according to claim 1, which is in the form of tablets, capsules, granules or pellets.

7. The pharmaceutical composition according to claim 1, wherein the PVAC and PVP in the coating (c) are in an amount of 1 to 30 wt %, based on the total weight of the pharmaceutical composition.

8. The pharmaceutical composition according to claim 7, wherein the PVAC and PVP in the coating (c) are in an amount of 2 to 20 wt %, based on the total weight of the pharmaceutical composition.

9. The pharmaceutical composition according to claim 1, wherein the layer thickness of the coating (c) is in the range of 10 to 50 μm.

10. The pharmaceutical composition according to claim 9, wherein the layer thickness of the coating (c) is in the range of 10 to 40 μm.

11. The pharmaceutical composition according to claim 10, wherein the layer thickness of the coating (c) is in the range of 10 to 25 μm.

12. The pharmaceutical composition according to claim 1, wherein the weight ratio of polyvinyl acetate to polyvinyl pyrrolidone in the coating (c) ranges from 1.5:1 to 14:1.

13. The pharmaceutical composition according to claim 12, wherein the weight ratio of polyvinyl acetate to polyvinyl pyrrolidone in the coating (c) ranges from 9:1 to 12:1.

14. The pharmaceutical composition according to claim 13, wherein the excipient (b) is an embedding material selected from the group consisting of polyvinyl pyrrolidone and cellulose ethers.

15. The pharmaceutical composition according to claim 14, wherein the embedding material (b) is a cellulose ether.

16. The pharmaceutical composition according to claim 14, wherein the weight ratio of Tamsulosin to embedding material (b) ranges from 1:3 to 1:25.

17. The pharmaceutical composition according to claim 16, wherein the weight ratio of Tamsulosin to embedding material (b) ranges from 1:4 to 1:19.

18. The pharmaceutical composition according to claim 17, wherein the core further comprises
one or more non-ionic surfactants selected from the group consisting of alkylglycosides, alkylmaltosides, alkylthioglucosides, polyoxyethylene alkyphenols, polyoxyethylene alkylethers, polyethyleneglycol fatty acid esters, polyethyleneglycol glycerol fatty acid esters, polyoxyethylene-polyoxypropylene block copolymers, polyglyceryl fatty acid esters, polyoxyethylene glycerides, polyoxyethylene vegetable oils, polyoxyethylene hydrogenated vegetable oils and sterols.

19. The pharmaceutical composition according to claim 18, wherein the non-ionic surfactant is a polyoxyethylene sorbitan fatty acid ester.

20. The pharmaceutical composition according to claim 19, wherein the weight ratio of Tamsulosin to the non-ionic surfactant ranges from 1:12 to 1:25.

21. The pharmaceutical composition according to claim 20, wherein the weight ratio of Tamsulosin to the non-ionic surfactant ranges from 1:16 to 1:19.

22. A sustained-release pharmaceutical composition, comprising Tamsulosin in the form of the base and/or in the form of a pharmaceutically acceptable salt, characterized in that the ratio between maximum plasma concentrations of the Tamsulosin determined after administration of said pharmaceutical composition under fasting conditions and under fed conditions according to the Cmax_{fasting/fed}-test is less than 1.35.

23. The pharmaceutical composition according to claim 22, wherein the ratio is less than 1.25.

24. The pharmaceutical composition according to claim 22, wherein the ratio is less than 1.2.

25. The pharmaceutical composition according to claim 22, wherein the ratio is equal to or less than 1.15.

26. A process for the manufacture of a pharmaceutical composition according to claim 25 comprising the steps of
(i) providing Tamsulosin, optionally in combination with at least one excipient (b) and
(ii) applying a coating (c) thereon which contains a combination of polyvinyl acetate and polyvinyl pyrrolidone.

27. The process according to claim 26, comprising the steps of
(i1) providing neutral pellets,
(i2) optionally coating said neutral pellets with a first subcoat,
(i3) coating said pellets of step (i1) or (i2) with a dispersion that contains Tamsulosin,
(i4) optionally coating said pellets of step (i3) with a second subcoat,
(ii1) applying on said pellets of steps (i3) or (i4) a coating which contains a combination of polyvinyl acetate and polyvinyl pyrrolidone, and
